(19) United States
(12) Patent Application Publication
(10) Pub. No.: US 2008/0033059 A1
(43) Pub. Date: Feb. 7, 2008

(54) Enteric Coating Compositions

(75) Inventors: Suresh Pareek, Mumbai (IN); Ashok Mohanty, Mumbai (IN); Shivaji Kamble, Mumbai (IN)

Correspondence Address: HAMRE, SCHUMANN, MUELLER & LARSON, P.C., P.O. BOX 2902, MINNEAPOLIS, MN, 55402-0902, US

(73) Assignee: Ideal Cures PVT. ltd., Mumbai (IN)

(21) Appl. No.: 10/589,862

(22) PCT Filed: Feb. 19, 2004
(86) PCT No.: PCT/IB04/00404

§ 371 Date: Apr. 30, 2007

(51) Int. Cl. A61K 47/14

(52) U.S. Cl. 514/772.6

(57) ABSTRACT

This invention is in the field of enteric film coating, dry powder compositions for coating pharmaceutical dosage forms. It relates to aqueous enteric coating and provides a dry enteric film coat composition based on an acrylate copolymer and other excipients, but without an alkalinizing agent in the composition.

### FIELD OF THE INVENTION

This invention is in the field of enteric film coating, dry powder compositions for use in making an aqueous enteric suspension, which may be used, for coating pharmaceutical dosage forms. It relates to aqueous enteric coating for preventing the release of ingredients of the dosage form in the gastric environment of the stomach and releasing the ingredients of the dosage form in the intestine. It concerns providing a dry enteric film coat composition based on an acrylate copolymer and other excipients, but without an alkalinizing agent in the composition, for use in making an aqueous enteric coating suspension that may be used in coating pharmaceutical dosage forms with a stomach insoluble coating that is soluble in the intestinal juices.

### DESCRIPTION OF PRIOR ART

Several aqueous enteric film coating systems based on cellulose acetate phthalate (AQUATERIC®), copolymers of acrylic acid esters and methacrylic acid (EUDRAGIT-L®) and polyvinylacetate phthalate (SURETERIC®) are known. The EUDRAGIT-L system is available as a powder (L100-55) and as an aqueous dispersion (L30-D). Both these products recommend a lot of processing steps for reconstitution, which involve addition of powder to water, alkalinizing it with a base, stirring for 30 minutes at medium speed, filtration, addition of plasticizer, and anti-foam agent, further stirring and final filtration. In addition if there is a slight deviation from the reconstitution as prescribed there is the formulation of a non-redispersable coagulum rendering the entire operation of no use. In case of AQUATERIC® the powder has to be dispersed in water followed by addition of plasticizer, surfactant and optional pigments to form the suspension.

The EUDRAGIT L30-D suspension is a dispersion of ethyl acrylate/methacrylic acid copolymer, 30% by weight in water and requires a multi-step process to form a complete aqueous dispersion including addition of plasticizer, separating agent, anti-foam, optionally pigments, stirring and filtration with similar special precautions to avoid formation of a coagulum.

The SURETERIC® composition of Colorcon described in U.S. Pat. No. 5,733,575 teaches the complete formulation of an enteric film coating pre-mix, readily water dispersible with anti-foam in two steps. However one has to add a viscosity modifier to prevent sedimentation during coating.

Lehmann et al. U.S. Pat. No. 4,520,172, which is incorporated herein by reference, discloses a binary mixture of EUDRAGIT L copolymer and a suitable alkalinizing agent or “salt-forming agent”.

Chittamuru et. al. U.S. Pat. No. 6,420,473 which is incorporated herein by reference discloses enteric film coating compositions in dry form based on copolymer of ethyl acrylate and methacrylic acid of the EUDRAGITL system analogous to the SURETERIC® system. Further the U.S. Pat. No. 6,420,473 discusses the importance of an alkalinizing agent capable of reacting with the acrylic resin such that after reaction 0.1 to 10 mole percent of the acidic groups in the resin are present in salt form.

### SUMMARY OF THE INVENTION

It is an object of this invention to provide a fully formulated, enteric film coating composition based on the copolymer of methacrylic acid copolymer without an alkalinizing agent to neutralize the free acids in the acrylate resin and is in a dry mix form readily dispersible in water, devoid of caking or agglomeration, capable of producing a film coat with good tensile strength to give at tack free coat.

Another object of this invention is to provide a fully formulated, enteric film coating composition based on the EUDRAGIT L copolymers that disperses in water without formation of coagulum.

Another object of this invention is to produce a dry mix enteric film coat composition that is devoid of any alkalinizing agent in the composition.

Thus this proves to be a superior dry mix in that any alkalinizing effects are avoided.

The use of alkalinizing agent is also to ensure stability of the dry mix during long-term storage, as the free carboxylic acid groups in the resin tend to have a slight destabilizing effect.

The composition of the present invention is such that there is no need for any alkalinizing agent and the dry mix remains stable even without the alkalinizing agent

When dispersed the free carboxylic acid groups get neutralized by the alkalinizing agent to form a salt. This is essential because if the amount of free carboxylic acid groups is not neutralized then there may be formation of an enteric coat on coating which is not resistant to the stomach environment.

The present invention achieves the same effect without the use of an alkalinizing agent and hence certainly represents an inventive step in the field of enteric coating dry powder compositions.

### DETAILED DESCRIPTION OF THE INVENTION

In accordance with the invention, the edible, non-toxic dry powder composition may be reconstituted to make an aqueous enteric suspension, which may be used for tablet coating comprises an acrylic resin (Kolicoat MAE 100P), a detackifier, an opacifier and pigments but devoid of any alkalinizing agent. Optionally other additives like a plasticizer, anti-agglomeration agent, a secondary film former, or a secondary detackifier may be added. A particularly preferred embodiment of this invention of dry powder composition contains a methacrylate copolymer, a detackifier, an opacifier and pigments but no alkalinizing agent.

The process of making the inventive dry powder composition comprises the steps of mixing the methacrylate copolymer, a detackifier, an opacifier, pigments and optionally a plasticizer, anti-agglomeration agent, a secondary film former, or a secondary detackifier. The resulting enteric film coating dry powder composition and anti-foam may be readily dispersed in water using a high-shear mixer to avoid coagulum formulation and is ready to use within 30 minutes.

In accordance with the invention, a method of coating substrates, such as pharmaceutical dosage forms comprises mixing sequentially the inventive dry composition devoid of an alkalinizing agent and an anti foam agent (if required) into water to form an enteric coating suspension that is ready to use and applying the coating suspension to form a film coat on the substrate and drying the film coat on the substrate.

The enteric coat or polymer is methacrylic acid copolymer preferably Type, C manufactured and sold by BASF under the tradename of Kolicoat MAE 100P which complies USP Pharmacopoeial requirements.

The free carboxylic acid groups present in the copolymer are such that they do not require to be neutralized with an alkalinizing agent for the preparation of the dry film coating composition.

Preferably the content of the methacrylic acid copolymer comprises about 30 to about 90% by weight of the dry coating composition of the invention.

There is no alkalinizing agent required in the present invention of dry film coating composition to react with the free carboxylic acid groups of the copolymer and thus none of the acidic groups are present in salt form.

The detackifier may be talc, kaolin, glyceryl monostearate or mixtures thereof and is used to reduce tablet to tablet sticking that may occur during the coating process. Preferably, the detackifier comprises about 7.5% to about 35% by weight of the dry coating composition of the invention.

The plasticizer may be diethylphthalate, triethylcitrate, polyethylene glycol having a molecular weight in the range of 200 to 8000, glycerol, castor oil or mixtures thereof Preferably, the plasticizer comprises about 5% to about 30% by weight of the dry coating composition of the invention.

The opacifier may be titanium dioxide, talc, magnesium carbonate or mixtures thereof Preferably, the opacifier comprises 0% to about 40% by weight of the inventive dry coating composition used.

Anti-agglomerating agents, which may be optionally included, are kaolin; secondary film formers include gums, alginates and cellulose derivatives, while a second detackifier used maybe an organic or inorganic salt or mixtures thereof.

It has been unexpectedly found that there is no “color bleeding” when lake pigments are added to this inventive dry film coating composition in spite of the absence of an alkalinizing agent in the composition.

A preferred process for manufacturing the inventive dry film coating composition is by conventional dry blending in a food processor or “V-blender” or a similar device. The aqueous homogenous suspensions ready for film coating are prepared by using a high shear mixer after dispersing the dry composition in deionised water. The following example illustrates the invention.

### EXAMPLE 1

Diclofenac Sodium Core (1.5 kg total charge, 50 mg Diclofenac Sodium per tablet) were coated with coating composition prepared as per formulation given below:

| Ingredient | Quantity taken | | Percentage (% w/w) |
|---|---|---|---|
| Kollicoat MAE 100 P | 97.5 | gms | 65 |
| Polyethylene glycol 6000 | 12 | gms | 08 |
| (Mfg. Clariant, Germany) | | | |
| Talcum Powder | 25.56 | gms | 17 |
| Titanium Dioxide | 15 | gms | 10 |
| Total | 150.00 | gms | 100.00% |

The inventive dry powder composition is Rapidly mixed for 30 seconds, the process repeated 3 times with impermanent mixing with spatula in food processor to form the Uniform Blend.

The Inventive Enteric Coating Suspension was then prepared by using simple propeller stirrer. 600 gms of Demineralized Water taken into a Beaker, Stir the DM water with the help of propeller stirrer to form a vortex. Add the 150 gms of above inventive dry powder mixture to vortex slowly to avoid lump formation. Stir suspension with medium speed for 45 minutes. The suspension will ready for Use.

In a 12 inch Conventional coating pan (make Bectochem Engineers) along with the Spray gun (Bullows-630) of 1 mm spray nozzle diameter. Peristaltic Pump (Electrolab-PP-201V) with silicone tubing of internal diameter 4 mm used. The tablets placed in coating pan & prewarmed for 10-15 minutes at 55° C. The tablets were coated keeping following parameters.

### Coating Process Parameters:

| | |
|---|---|
| Fluid Delivery Rate (Spray rate in g/min.) | 5 |
| Atomization Air Pressure (Kg/cm²) | 0.5 |
| Inlet air Temp. (Deg. Celsius) | 55 |
| Tablet Bed Temperature (Deg. Celsius) | 30-32 |
| Pan Speed (rpm) | 18 |

No tackiness or Tablet to Tablet Sticking was observed during whole coating process. Tablets post dried in pan for 10 minutes at low rpm. Then Tablets were dried at 40° C. for 2 hours in Hot air Dryer-Oven Type for curing.

The coated tablets were evaluated using USP Dissolution Method <711> according to “Delayed-release” diclofenac tablets monograph. As prescribed by this method, six tablets coated as described in Example 1 were placed in 0.1N HCl for 2 hours at 37° C. the release in acid phase of the test after 2 hours was 0.1% as compared with the upper limit of 10%. The six tablets were then placed in Phosphate buffer (pH=6.8), the amount of Diclofenac Sodium released in 45 minutes was greater than 85% within 25 minutes, as compared to compendial requirement of not less than 75% released after 45 Minutes.

The final coated tablets were also evaluated using a modified version of USP Disintegration Method <701>. Fifty tablets prepared as described in Example 1 were stressed for 100 revolutions in a friabilator. Then, the 50 stressed tablets were placed in a basket assembly and immersed for one hour in simulated gastric fluid (0.1 N HCl). The basket was moved up and down in the simulated gastric fluid at a rate of about 28-32 cycles/minute. Fifty unstressed tablets were also placed in a basket assembly and immersed for one hour in simulated gastric fluid. The basket was moved up and down at a rate of about 28-32 cycles/min. The integrity of the tablets was evaluated after removal from the simulated gastric fluid. In both cases (stressed and unstressed), none of the tablets exhibited signs of bloating, cracks or fissures. The final coated tablets were also examined qualitatively. The resulting orange coating was smooth and uniform and showed no evidence of chipping, peeling or color non-uniformity.

### EXAMPLES 2-4

Examples 2-4 are inventive dry powder film coating compositions without alkalinizing agent wherein the concentration of the methacrylate copolymer is varied in the dry coating composition the other components and their concentration being constant. The process parameters for coating remained the same except for very minor changes like adjustment of temperature or atomization air pressure were used if required. Diclofenac Sodium Core (1.5 kg total charge, 50 mg Diclofenac Sodium per tablet) were coated with coating composition prepared as per formulation given below:

Comparative quantitative compositions Examples 2-4
Weight % in 150 grams

| Ingredients | Example 2 | Example 3 | Example 4 |
|---|---|---|---|
| Kollicoat MAE 100 P | 40 | 50 | 69 |
| Polyethylene glycol 6000 | 25 | 23 | 15 |
| Talcum Powder | 22 | 17 | 12 |
| Titanium Dioxide | 10 | 08 | 3 |
| Lake Pigment (Color orange) | 3 | 2 | 1 |

After coating the tablets with the compositions as given in examples 2 to 4, the enteric-coated tablets were again subjected to the USP tests as in example 1.

It was observed that the percentage of tablets passing the standard disintegration test and stressed disintegration test were more than 95% in case of all the examples of the dry powder film coating compositions mentioned in Examples 2 to 4.

We claim:

1. A non-toxic, edible, enteric film coating, dry powder composition for use in preparing an aqueous enteric coating suspension which may be used, in coating of substrates comprising:
a. A methacrylate copolymer
b. A plasticizer
c. A film coating detackifier
d. An opacifier
e. Optionally pigments (FD&C and D&C lakes) which are approved for use for human consumption
Wherein the dry powder composition does not contain any alkalinizing agent.

2. The enteric film coating dry composition of claim 1, the methacrylate copolymer being preferably of Type C.

3. The enteric film coating dry composition of claim 1, comprising from about 20-90% of the methacrylate copolymer by weight of the composition, preferably from 40-75% by weight of the composition.

4. The enteric film coating dry composition of claim 1, comprising a plasticizer, preferably polyethylend glycol 6000.

5. The enteric film coating dry composition of claim 1, comprising from about 5-30% of the plasticizer by weight of the composition, preferably from 5-25% by weight of the composition.

6. The enteric film coating dry composition of claim 1, comprising of a film detackifier, preferably talcum.

7. The enteric film coating dry composition of claim 1, comprising from about 7.5-35% of the film detackifier by weight of the composition, preferably from 10-30% by weight of the composition.

8. The enteric film coating dry composition of claim 1, comprising of an opacifier, preferably titanium dioxide.

9. The enteric film coating dry composition of claim 1, comprising from about 0.1-40% of an opacifier by weight of the composition, preferably from 2.5-30% by weight of the composition.

10. The enteric film coating dry composition of claim 1, optionally comprising of pigments, preferably FD&C and D&C lakes or mixtures thereof.

11. The enteric film coating dry composition of claim 1, optionally comprising from about 0-50% of a pigment by weight of the composition.

12. A process of making a dry powder enteric film coating composition which may be reconstituted for obtaining an aqueous enteric suspension used for coating of substrates comprising of dry blending of the following ingredients:
a. A methacrylate copolymer
b. A plasticizer
c. A film detackifier
d. An opacifier
e. Optionally pigments
In a suitable mixer or food processor to achieve a uniform mix of the dry powder film coating composition.
